(19) United States
(12) Patent Application Publication
(10) Pub. No.: US 2026/0183104 A1
(43) Pub. Date: Jul. 2, 2026

(54) PROSTHETIC HEART VALVE

(71) Applicant: Edwards Lifesciences Corporation, Irvine, CA (US)

(72) Inventors: Tamir S. Levi, Zikhron Yaakov (IL); Karin Lavon, Ra'anana (IL)

(73) Assignee: Edwards Lifesciences Corporation, Irvine, CA (US)

(21) Appl. No.: 19/543,741

(22) Filed: Feb. 18, 2026

Related U.S. Application Data
(63) Continuation of application No. PCT/US2024/045201, filed on Sep. 4, 2024.
(60) Provisional application No. 63/536,669, filed on Sep. 5, 2023.

(51) Int. Cl. A61F 2/24

(52) U.S. Cl. CPC A61F 2/2418; A61F 2220/0075; A61F 2250/0037

(57) ABSTRACT

A prosthetic valve includes a frame having an inflow end and an outflow end, and a plurality of leaflets. The frame includes a first row of angled struts defining the outflow end, a second row of angled struts upstream of the first row of angled struts, a third row of angled struts upstream of the second row of angled struts, a plurality of first axial frame members bridging the first and second rows of angled struts, and a plurality of second axial frame members bridging the second and third rows of angled struts. Each leaflet comprises an outflow edge, a cusp edge, and two opposing side portions. Each side portion of each leaflet is attached to one of the first axial frame members and one of the second axial frame members.

### CROSS-REFERENCE TO RELATED APPLICATIONS

This application is a continuation of International Patent Application No. PCT/US 2024/045201, filed Sep. 4, 2024, which claims the benefit of U.S. Provisional Application No. 63/536,669, filed Sep. 5, 2023, all of which applications are incorporated by reference herein in their entireties.

### FIELD

The present disclosure relates to implantable, radially expandable prosthetic devices, such as prosthetic heart valves, and to methods, assemblies, and apparatuses for delivering, expanding, implanting, and deploying such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve (or simply “prosthetic valve”) can be mounted in a crimped state on the distal end of a delivery apparatus and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size. Despite the recent advancements in percutaneous valve technology, there remains a need for improved transcatheter prosthetic valves.

### SUMMARY

The present disclosure relates to methods and devices for treating valvular diseases. Specifically, the present disclosure is directed to implantable, radially expandable prosthetic devices, such as prosthetic heart valves, and to methods, assemblies, and apparatuses for delivering, expanding, implanting, and deploying such prosthetic devices.

A prosthetic valve can include a radially expandable and compressible frame comprising an inflow end and an outflow end, and a leaflet assembly comprising a plurality of leaflets secured to the frame. In addition to these features, a prosthetic valve can further comprise one or more of the components disclosed herein.

In certain examples, the annular frame includes a first row of angled struts defining the outflow end, a second row of angled struts upstream of the first row of angled struts, a third row of angled struts upstream of the second row of angled struts, a plurality of first axial frame members bridging the first row of angled struts and the second row of angled struts, and a plurality of second axial frame members bridging the second row of angled struts and the third row of angled struts.

In some examples, the first row of angled struts, the second row of angled struts, and the first axial frame members define a first row of first cells. In some examples, the first cells are six-sided cells. In some examples, the second row of angled struts, the third row of angled struts, and the second axial frame members define a second row of second cells upstream of the first row of first cells. In some examples, the second cells are six-sided cells.

In some examples, each leaflet includes an outflow edge, a cusp edge opposite the outflow edge, and two opposing side portions extending between the outflow edge and the cusp edge. In some examples, selected ones of the first axial frame members include first leaflet attachment frame members and selected ones of the second axial frame members include second leaflet attachment frame members. In some examples, each side portion of each leaflet can be attached to one of the first leaflet attachment frame members and one of the second leaflet attachment frame members.

In some examples, the annular frame includes a first row of angled struts defining the outflow end of the frame, a second row of angled struts upstream of the first row of angled struts, a third row of angled struts upstream of the second row of angled struts, a fourth row of angled struts upstream of the third row of angled struts, a plurality of first axial frame members bridging the first row of angled struts and the second row of angled struts, and a plurality of second axial frame members bridging the second row of angled struts and the third row of angled struts. In some examples, selected ones of the first axial frame members include first leaflet attachment frame members and selected ones of the second axial frame members include second leaflet attachment frame members.

In some examples, each leaflet include an outflow edge, a curved cusp edge opposite the outflow edge, and two opposing side portions extending between the outflow edge and the cusp edge. In some examples, each side portion of each leaflet is attached to one of the first leaflet attachment frame members along a majority of a length thereof and to one of the second leaflet attachment frame members along a majority of a length thereof. In some examples, the cusp edge of each leaflet extends from a first location at an upstream end of one of the second leaflet attachment frame members to a second location at an upstream end of another one of the second leaflet attachment frame members.

In some examples, the annular frame includes a first row of first cells defining the outflow end of the frame, a second row of second cells upstream of the first row of first cells, and a third row of third cells upstream of the second row of second cells.

In some examples, the first cells are six-sided cells. In some examples, the second cells are six-sided cells and are smaller than the first cells. In some examples, the third cells are four-sided cells and are smaller than the second cells.

Certain aspects of the disclosure concern a method including delivering a prosthetic device in a radially compressed state to a target location, and radially expanding the prosthetic device to a radially expanded state. The prosthetic device can be any one of the prosthetic valves described above.

The above method can be performed on a living animal or on a simulation, such as on a cadaver, cadaver heart, anthropomorphic ghost, simulator (for example, with body parts, heart, tissue, etc. being simulated).

In some examples, a prosthetic valve comprises one or more of the components recited in Examples 1-47 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of a portion of the frame of FIG. 2, showing the portion of the frame in a straightened (non-annular) state.

FIG. 4 is a side view of an exemplary delivery apparatus configured to deliver and implant a radially expandable prosthetic valve at an implantation site.

FIG. 5 is a flattened view of a leaflet, according to one example.

FIG. 6A is a cross-sectional view of a portion of a frame and a leaflet structure showing adjacent commissure tabs of two leaflets of the type shown in FIG. 5 secured to a corresponding commissure window of the frame, according to one example.

FIG. 6B is side view of a commissure tab of the leaflet of FIG. 5 when secured to a corresponding commissure window of a frame, according to one example.

FIG. 7A is a top view of a prosthetic valve having a leaflet structure, wherein the leaflet structure is shown in a closed state, according to one example.

FIG. 7B is a top view of the prosthetic valve of FIG. 7A in which the leaflet structure is shown in a partially open state, according to one example.

FIG. 7C is a top view of the prosthetic valve of FIG. 7A in which the leaflet structure is shown in a fully open state, according to one example.

FIG. 8 is a flattened view of a leaflet, according to one example.

FIG. 9 is a side view of a portion of a frame to which the leaflet of FIG. 8 can be attached, according to one example.

FIG. 10 is a side view of a portion of a frame to which the leaflet of FIG. 8 can be attached, according to another example.

FIG. 11 is a side view of a portion of a frame to which the leaflet of FIG. 8 can be attached, according to a further example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. Further, as used herein, “and/or” means “and” or “or,” as well as “and” and “or.”

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

### Overview of Prosthetic Valves

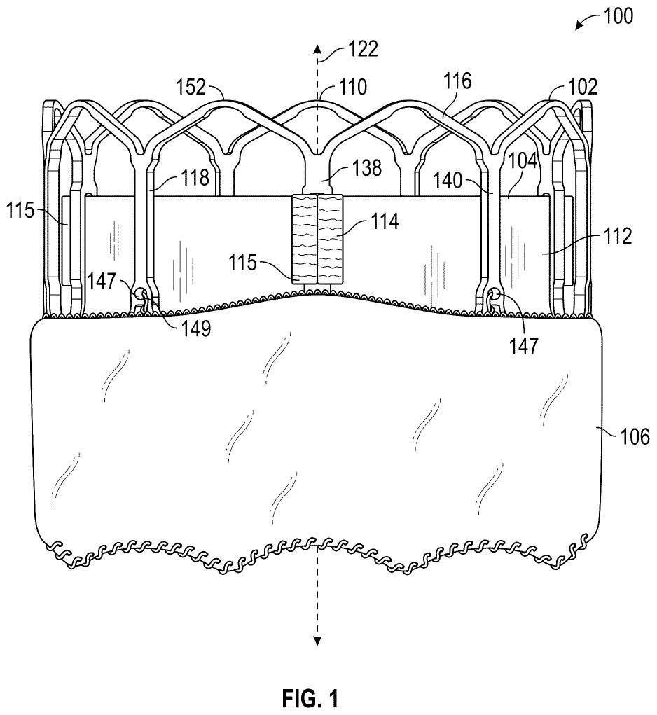

FIG. 1 shows a prosthetic heart valve 100 (or simply, “prosthetic valve”), according to one example. Any of the prosthetic valves disclosed herein can be adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include an annular stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (for example, three leaflets, as shown in FIG. 1), collectively forming a leaflet structure (also referred to as a “leaflet assembly”), which can be arranged to collapse in a tricuspid arrangement. The leaflets 112 can be secured to one another at their adjacent sides (for example, commissure tabs 115) to form commissures 114 of the valvular structure 104. For example, each leaflet 112 can comprise opposing commissure tabs 115 disposed on opposite sides of the leaflet 112 and a cusp edge portion extending between the opposing commissure tabs. The cusp edge portion of the leaflets 112 can have an undulating, curved scalloped shape, and can be secured directly to the frame 102 (for example, by sutures). However, in alternate examples, the cusp edge portion of the leaflets 112 can be secured to an inner skirt (disposed on an inner surface of the frame 102) or a fabric reinforcing member which is then secured to the frame 102. In some examples, the leaflets 112 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein. Example structures of leaflets 112 are described more fully below.

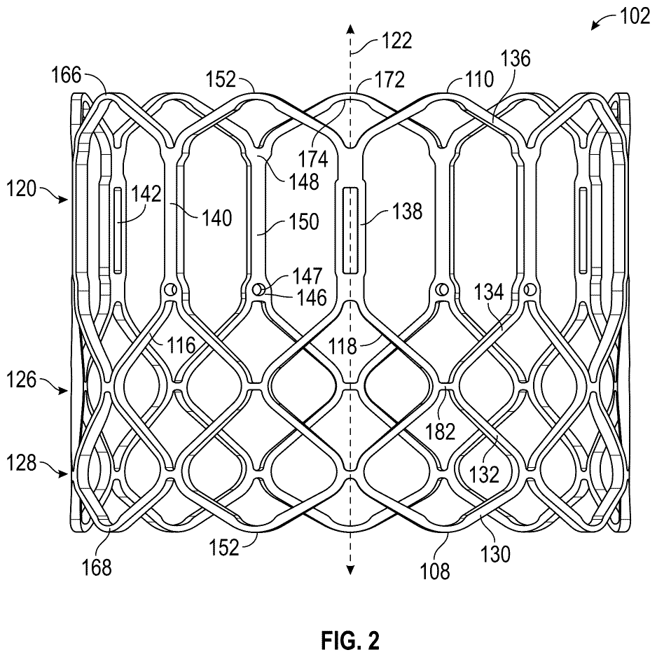

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to some angled struts (for example, the angled struts 134 of FIG. 2). Examples of such outer skirts, as well as various other outer skirts, that can be used with the frame 102 can be found in U.S. provisional Ser. No. 63/366,599 filed Jun. 17, 2022, which is incorporated by reference herein.

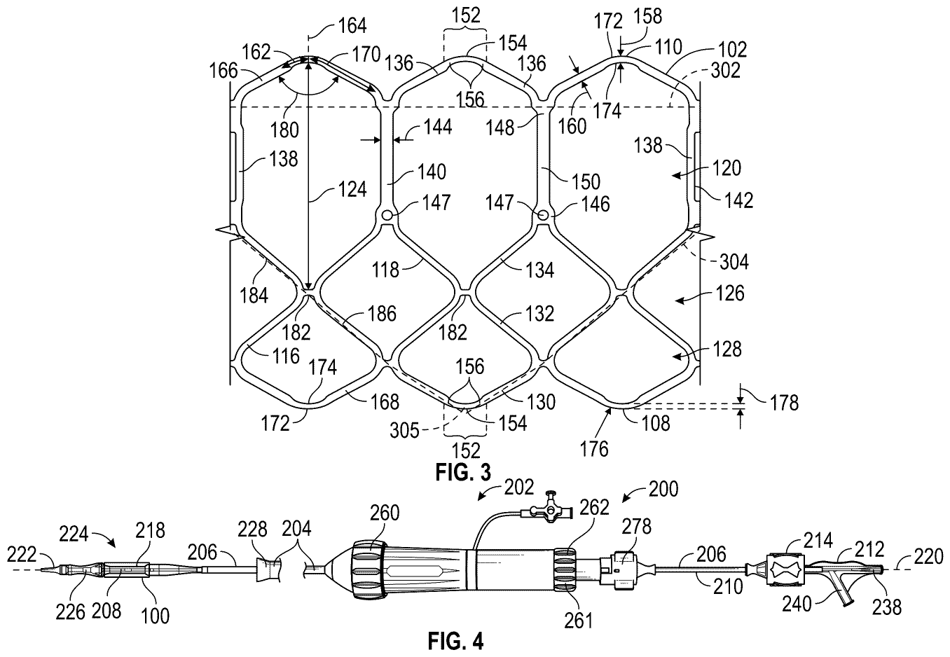

The frame 102 can be radially compressible and expandable between a radially compressed (or collapsed) configuration and a radially expanded configuration (the expanded configuration is shown in FIG. 1). The frame 102 is shown alone in FIG. 2 and a portion of the frame 102 in a straightened (non-annular) configuration is shown in FIG. 3.

The frame 102 can be made of any of various suitable plastically-expandable materials (for example, stainless steel, etc.) or self-expanding materials (for example, nickel titanium alloy (NiTi), such as nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIGS. 2-3, the frame 102 can comprise a plurality of interconnected struts 116 which form multiple rows of open cells 118 between the outflow end 110 and the inflow end 108 of the frame 102. In some examples, the frame 102 can comprise three rows of cells 118 including a first (the upper row in the orientation shown in FIGS. 2-3) row 120 of cells 118 disposed at the outflow end 110, a second row 126 of cells 118 upstream of the first row 120 of cells, and a third row 128 of cells 118 at the inflow end 108 of the frame 102. The first row 120 comprises cells 118 that are elongated in an axial direction (relative to a central longitudinal axis 122 of the frame 102), as compared to cells 118 in the remaining rows of cells. For example, the cells 118 of the first row 120 of cells can have a longer axial length 124 than cells 118 in the second row 126 of cells and the third row 128 of cells.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (for example, four or five) and/or more or less than nine cells per row. In some examples, the cells 118 in the first row 120 of cells may not be elongated compared to cells 118 in the remaining rows of cells of the frame 102 (for example, the second row 126 of cells and the third row 128 of cells).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 136 arranged end-to-end and extending circumferentially at the outflow end 110 of the frame; a second row of circumferentially extending, angled struts 134; a third row of circumferentially extending, angled struts 132; and a fourth row of circumferentially extending, angled struts 130 at the inflow end 108 of the frame 102.

In the depicted example, the first row of angled struts 136 is upstream of the second row of angled struts 134, which is upstream of the third row of angled struts 132, which is upstream of the fourth row of angled struts 130. As described herein, a frame component (for example, a row of angled struts) is deemed to be upstream of a reference object (for example, another row of angled struts) if the frame component is closer to the inflow end 108 (or farther away from the outflow end 110) than the reference object.

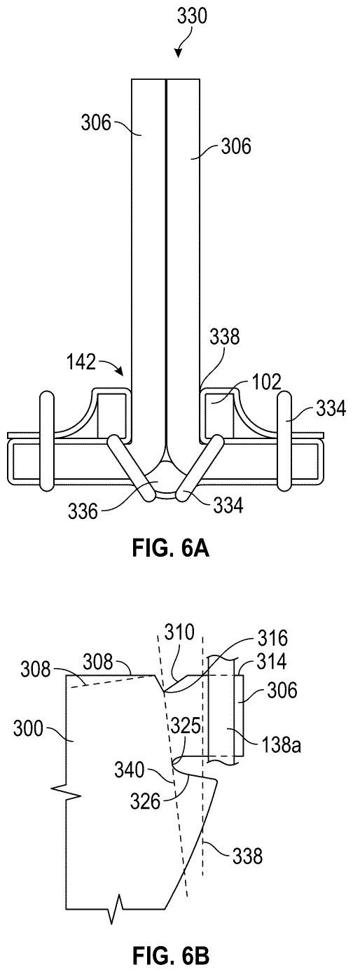

The two rows of angled struts that are closest to the outflow end 110 (for example, the first row of angled struts 136 and the second row of angled struts 134) can be connected by a plurality of axial (or axially extending) struts 140. Some of the axial struts 140 can define commissure windows 142 (for example, open windows extending through a thickness of the axial struts). As described herein, such axial struts with commissure windows 142 can also be referred to as axially extending window struts 138a, 138b (or simply “window struts”). Each of the windows 142 comprise a pair of window struts 138a, 138b (FIG. 6A) that are be spaced apart from one another around the frame 102, in a circumferential direction. Each commissure window 142 can be adapted to receive a pair of commissure tabs of a pair of adjacent leaflets 112 arranged into a commissure (for example, commissure 114 shown in FIG. 1). In some examples, the commissure windows 142 and/or the window struts 138 defining the commissure windows 142 can be referred to as commissure features or commissure supports. Each commissure feature or support is configured to receive and/or be secured to a pair of commissure tabs of a pair of adjacent leaflets.

One or more (for example, two, as shown in FIGS. 2-3) axial struts 140 without commissure windows 142 can be positioned between, in the circumferential direction, two commissure supports comprising the commissure windows 142. Since the frame 102 can include fewer cells per row (for example, nine) and fewer axial struts 140 between each pair of window struts 138, as compared to some more traditional prosthetic heart valves, each cell 118 can have an increased width (in the circumferential direction), thereby providing a larger opening for blood flow and/or coronary access.

In certain examples, each axial strut 140 (including each window strut 138) can extend from a location defined by the convergence of the lower ends (for example, ends arranged inward of and farthest away from the outflow end 110) of two angled struts 136 (which can also be referred to as an upper strut junction or upper elongated strut junction) to another location defined by the convergence of the upper ends (for example, ends arranged closer to the outflow end 110) of two angled struts 134 (which can also be referred to as a lower strut junction or lower elongate strut junction). Each axial strut 140 (including each window strut 138) thus forms an axial side of two adjacent cells of the first row 120 of cells 118.

In some examples, as shown in FIG. 3, each axial strut 140 can have a width 144 that is larger than a width of the angled struts 130, 132, 134, and 136. As used herein, a “width” of a strut is measured between opposing locations on opposing surfaces of the strut that extend between the radially facing inner and outer surfaces of the strut (relative to the central longitudinal axis 122 of the frame 102). A “thickness” of a strut is measured between opposing locations on the radially facing inner and outer surfaces of the strut and is perpendicular to the width of the strut. In some examples, the width 144 of the axial struts 140 is 50-200%, 75-150%, or at least 100% larger than (for example, double) the width of the angled struts of the frame 102.

By providing the axial struts 140 with a width 144 that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width compared to alternate prosthetic valves that have more than nine cells per row, the wider axial struts 140 can be more easily incorporated into the frame 102, without sacrificing open space for blood flow and/or coronary access.

As described above, commissure tabs 115 of adjacent leaflets 112 can be secured together to form commissures 114 (FIG. 1). Each commissure 114 of the prosthetic valve 100 comprises two commissure tabs 115 paired together, one from each of two adjacent leaflets 112, and extending through a commissure window 142 of the frame 102. Each commissure 114 can be secured to a respective window strut 138 forming commissure window 142.

The cusp edge portion (for example, scallop edge) of each leaflet 112 can be secured to the frame 102 via one or more fasteners (for example, sutures). In some examples, the cusp edge portion of each leaflet 112 can be secured directly to the struts of the frame 102 (for example, angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt disposed on an inner surface of the frame, and the inner skirt can then be secured directly to the frame 102.

Various methods for securing the leaflets 112 to a frame, such as the frame 102, are disclosed in U.S. provisional patent applications 63/278,922, filed Nov. 12, 2021, and 63/300,302, filed Jan. 18, 2022, both of which are incorporated by reference herein.

As shown in FIGS. 2-3, in some examples, one or more of or each of the axial struts 140 can comprise an inflow end portion 146 (for example, an end portion that is closest to the inflow end 108) and an outflow end portion 148 that are widened relative to a middle portion 150 of the axial strut 140. In some instances, the inflow end portion 146 of the axial strut 140 can comprise an aperture 147. The apertures 147 can be configured to receive fasteners (for example, sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (for example, sutures), as shown in FIG. 1.

The interconnected struts 116 can also comprise horizontal struts 182 that extend between adjacent cells 118 of a row of cells of the frame 102 (FIGS. 2-3). The horizontal struts 182 can extend in a circumferential direction and also be referred to as circumferentially extending struts. The horizontal struts 182 can connect angled struts of two adjacent rows of angled struts of the frame 102 to one another. For example, each horizontal strut 182 can connect to two angled struts of one row of struts (for example, struts 134 shown in FIG. 3) and two angled struts in another, adjacent row of struts (for example, struts 132 shown in FIG. 3). As a result, an angled strut 184 extending between a window strut 138 and a horizontal strut 182 and an angled strut 186 extending between the horizontal strut 182 and another horizontal strut 182 disposed adjacent to the inflow end 108 of the frame 102 can be aligned along an angled line that can follow a scallop line of the leaflets (when the leaflets are attached to the frame 102). Thus, the horizontal struts 182 can allow the angled struts (for example, 184, 186) to follow a shape that more closely matches a shape of the scallop line of the leaflets when the frame 102 is in the radially expanded configuration (as shown in FIGS. 2-3). Additionally, the horizontal struts 182 can serve as spacers that can maintain a specified gap between the angled struts when the frame 102 is in the radially compressed configuration, thereby reducing a risk of pinching the leaflets between the struts in the radially compressed configuration.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

In certain examples, as shown in FIG. 3, each apex region 152 can comprise an apex 154 (the highest or most outward extending, in an axial direction, point) and two thinned (or narrowed) strut portions 156, one thinned strut portion 156 extending from either side of the apex 154 to a corresponding, wider, angled strut 136 (at the outflow end 110) or angled strut 130 (at the inflow end 108). In this way, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152.

The thinned strut portions 156 of the apex regions 152 can have a width 158 that is smaller than a width 160 of the angled struts 130 or 136 (FIG. 3). In some examples, the width 158 can be a uniform width (for example, along an entire length of the strut portion 156). In some examples, the width 158 of the thinned strut portions 156 can be from about 0.06-0.15 mm smaller than the width 160 of the angled struts 130 and/or 136.

Each of the thinned strut portions 156 of the apex regions 152 can have a length 162 in a range of 0.8-1.4 mm, 0.9-1.2 mm, 0.95-1.05 mm, or about 1.0 mm (for example, ±0.03 mm). In alternate examples, the length 162 is in a range of 0.3-0.7 mm, 0.4-0.6 mm, 0.45-0.55 mm, or about 0.5 mm (for example, ±0.03 mm). Because each outflow apex region 152 can include two thinned strut portions 156 having the same length 162, a total length of the apex region 152 can be two times the length 162.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152. One half the total length of each outflow strut 166 and inflow strut 168 is shown in FIG. 3 as length 170, which extends from an end of one angled strut 136 or 130 to the central longitudinal axis 164. Thus, the length of each outflow strut 166 and inflow strut 168 is two times length 170. In some examples, the length 170 for half of each inflow strut 168 can be different than the length 170 for half of each outflow strut 166.

In some instances, the length of each thinned strut portion 156 can be at least 25% of the length 170 of the corresponding half outflow strut 166 or inflow strut 168. Said another way, the length of each apex region 152 (a total length being two times the length 162) can be at least 25% of the total length (two times length 170) of the outflow strut 166 or inflow strut 168. In some examples, the length of each apex region 152 can be more than 25% of the total length of the corresponding outflow strut 166 or inflow strut 168, such as 25-35%.

In some examples, each apex region 152 can comprise a curved, axially facing outer surface 172 and an arcuate or curved, axially facing inner depression 174 which forms the thinned strut portions 156. For example, the curved inner depression 174 can depress toward the curved outer surface 172 from an inner surface of the angled strut portions 156, thereby forming the smaller width thinned strut portions 156. Thus, the curved inner depressions 174 can be formed on a cell side of the apex region 152 (for example, as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152 (for example, the curved outer surface 172 can have a constant curvature).

Each apex region 152 can have a radius of curvature 176, along the curved outer surface 172 (for example, in some instances, along an entirety or an entire length of the curved outer surface 172) (FIG. 3). In some instances, the radius of curvature 176 at the apex 154 and/or along the entire curved outer surface 172 of the apex region 152 can be greater than 1 mm. In some instances, the radius of curvature 176 can be in a range of 1-20 mm, 3-16 mm, or 8-14 mm. In some instances, the radius of curvature 176 can be greater than 10 mm. The radius of curvature 176 can be dependent on (and thus change due to changes in) the width 158 (for example, the amount of reduction in width from the angled struts 130 or 136) and the length 162 of the thinned strut portions 156.

Further, a height (an axial height) 178 of the apex regions 152, which can be defined in the axial direction from an outer surface of the two angled struts 130 or 136 to the curved outer surface 172 of the apex region 152 at the apex 154, can be the width 158 of the thinned strut portions 156 (FIG. 3). In this way, the height 178 of the apex regions 152 can be relatively small and not add much to the overall axial height of the radially expanded frame 102. Thus, the leaflets 112 secured to the frame 102 (FIG. 1) can be disposed close to the inflow end 108, thereby leaving a larger open space at the outflow end 110 of the frame 102 that is not blocked by the leaflets 112.

In some examples, each of the apex region 152 can form an angle 180 between the two angled struts 130 or 136 extending from either side of the corresponding apex region 152 (FIG. 3). In some instances, the angle 180 can be in a range of 120 (not inclusive) to 140 degrees (for example, such that the angle 180 is greater than 120 degrees and less than or equal to 140 degrees).

Additional details and examples of frames for prosthetic heart valves that include apex regions can be found in PCT Application No. PCT/US2022/025687, which is incorporated by reference herein.

### Exemplary Delivery Apparatus

FIG. 4 shows a delivery apparatus 200, according to an example, that can be used to implant an expandable prosthetic valve (for example, the prosthetic valve 100 and/or any of the other prosthetic valves described herein). In some examples, the delivery apparatus 200 can be specifically adapted for use in introducing a prosthetic valve into a heart.

The delivery apparatus 200 in the illustrated example of FIG. 4 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222, or to an alternate component at the distal end of the delivery apparatus 200 (for example, a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic valve in a radially compressed state. For example, as shown schematically in FIG. 4, a prosthetic device, such as the prosthetic valve 100, can be mounted around the balloon 218, at the valve mounting portion 224 of the delivery apparatus 200.

The balloon shoulder assembly, including the distal shoulder 226, can be configured to maintain the prosthetic valve 100 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 100 is mounted in the radially compressed state on the valve mounting portion 224 and during delivery of the prosthetic valve to the target implantation site. As such, the distal tip portion 228 can be configured to resist movement of the prosthetic valve 100 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 100.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn can be operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 can be configured to adjust the axial position of the intermediate shaft 206 relative to the outer shaft 204 (for example, for fine positioning at the implantation site). Further details on the delivery apparatus 200 can be found in PCT Application No. PCT/US2021/047056, which is incorporated by reference herein.

Although the delivery apparatus 200 depicted in FIG. 4 is specifically adapted to deliver a balloon expandable prosthetic valve, it is to be understood that variants of the delivery apparatus 200 can be adapted for delivery of self-expandable prosthetic valves and/or mechanically expandable prosthetic valves, as described in references incorporated above.

### Exemplary Leaflets for Prosthetic Valves

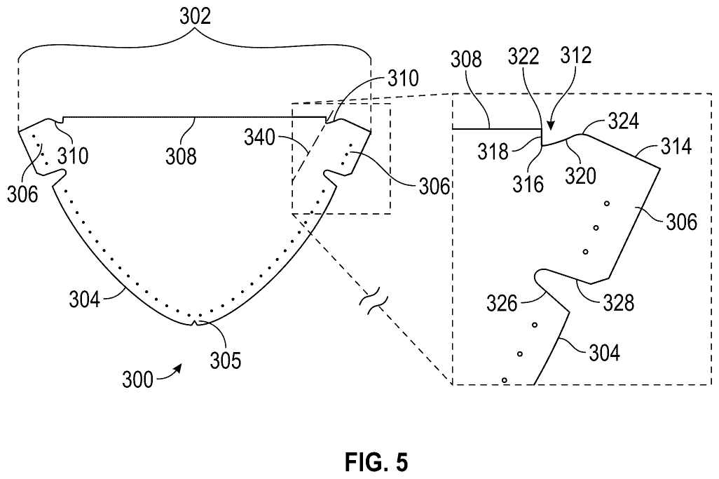

FIG. 5 shows a flattened view of a leaflet 300, according to one example. The leaflet 300 has an outflow edge 302, a cusp edge 304 opposite the outflow edge 302, and a pair of commissure tabs 306 extending from opposite sides of the leaflet 300 and between the outflow edge 302 and the cusp edge 304.

As shown, the outflow edge 302 of the leaflet 300 comprises a coaptation edge region 308 and two cut-out regions 310 (which can also be referred to as discontinuities in some examples) between opposite ends of the coaptation edge region 308 and the commissure tabs 306. In the depicted example, the cut-out regions 310 comprise V-shaped notches 312 that are adjacent to corresponding commissure tabs 306. In other words, the coaptation edge region 308 extends between the two notches 312.

Each commissure tab 306 has an outflow edge 314. The coaptation edge region 308 can be straight or substantially straight. When the leaflet 300 is flattened (prior to assembly with other leaflets and mounted to a frame), the outflow edge 314 of each commissure tab 306 can be angled relative to the coaptation edge region 308, for example, downwardly as shown in FIG. 5 (that is, toward the inflow end of the frame when mounting the leaflet to the frame).

Each notch 312 has a nadir 316, a first edge 318 extending from the nadir 316 to the coaptation edge region 308, and a second edge 320 extending from the nadir 316 to the outflow edge 314 of the corresponding commissure tab 306. In some examples, as depicted in FIG. 5, the first edge 318 is shorter than the second edge 320. In other examples, the first edge 318 can be about the same or longer than the second edge 320.

In certain examples, the first edge 318 can intersect the coaptation edge region 308 at a sharp-angled corner 322. In certain examples, the first edge 318 and the coaptation edge region 308 can form an angle of 90 degrees or greater (about 90 degrees in the example depicted in FIG. 5).

In certain examples, the second edge 320 can intersect the outflow edge 314 of the adjacent commissure tab 306 at a rounded or smoothed corner 324. In some examples, the second edge 320 and the outflow edge 314 of the adjacent commissure tab 306 can form an obtuse angle.

In certain examples, each commissure tab 306 is connected to the cusp edge 304 by a recessed region 326. For example, each commissure tab 306 can have an inflow edge 328 that is generally parallel to the outflow edge 314. The recessed region 326 can include a U-shaped or V-shaped notch formed between the cusp edge 304 and the inflow edge 328 of the commissure tab 306.

A plurality of leaflets 300 can be assembled together to form a leaflet assembly that is coupled to a frame of a prosthetic valve. For example, a prosthetic valve 100 (FIG. 1) can include a plurality of leaflets 300 instead of leaflets 112. As described above, to form the commissures of the leaflet assembly, each commissure tab 306 can be paired with an adjacent commissure tab of an adjacent leaflet 300 to form a commissure of a leaflet assembly that is connected to a frame. The leaflet assembly is movable between an open state which permits blood flow from the inflow end to the outflow end of the frame and a closed state which blocks blood fluid flow from the outflow end to the inflow end of the frame.

For example, FIG. 6A shows a commissure 330 comprising a pair of commissure tabs 306 of two adjacent leaflets that are inserted through a commissure window 142 defined between two struts 138a, 138b of the frame 102. One of the commissure tabs 306 is wrapped partially around one strut 138a on the outside of the frame 102 and the other commissure tab 306 is wrapped partially around the other strut 138b on the outside of the frame. The width of the commissure window 142 can be about the same or slightly smaller than the thickness of two commissure tabs 306 so that the pair of commissure tabs 306 are squeezed against each other inside the commissure window 142 and are tightly coupled together.

The commissure 330 can further include a reinforcing member 335, such as in the form of a piece of fabric, that assists in securing the commissure tabs 306 to the struts 138a, 138b of the frame 102. The reinforcing member 335 can comprise, for example, a piece of woven polyethylene terephthalate (PET) fabric, although other synthetic and/or natural materials can be used. The reinforcing member 335 has opposing end portion, each of which is folded to form an inner layer 335a, a first outer layer 335b, and a second outer layer 335c. Each end portion of the reinforcing member 335 is wrapped around a respective strut 138a or 138b to form a respective inner layer 335a and a respective first outer layer 335b that encloses the strut. Each second outer layer 335c extends an outer edge of a respective commissure tab 306 and along the outer surface of the commissure tab 306 where it is connected to the other second outer layer.

The reinforcing member 335 and the commissure tabs 306 can be secured to each other with one or more sutures to retain the commissure 330 on the struts 138a, 138b. For example, a pair of first suture lines 334 can be used to secure portions of the commissure tabs 306 closest to the commissure window 142. Each suture line 334 can be formed from a suture that forms stitches (for example, in-and-out stitches) that extend through a first outer layer 335b, a commissure tab 306, and a second outer layer 335c. A pair of second suture lines 337 can be used to secure the outer edge portions of the commissure tabs 306 to the reinforcing member 335. Each suture line 337 can be formed from a suture that forms stitches (for example, in-and-out stitches) that extend through an inner layer 335a, a first outer layer 335b, a commissure tab 306, and a second outer layer 335c.

In certain examples, the commissure 330 can further include a post or a wedge member 336 situated between the pair of commissure tabs 306 on the outside of the commissure window. In certain examples, the wedge member 336 can comprise a non-metallic material, such as a rope or a braided suture, such as an Ethibond suture (for example, a 2-0 Ethibond suture). The wedge member 336 can be retained against the commissure tabs 306 with the stitches of the first suture lines 334. For example, the stitches of each suture line 334 can extend through the wedge member 336 (in addition to the layers 335b, 335c of the reinforcing member 335 and the commissure tab 306).

The reinforcing member 335 and the wedge member 336 can further increase the stiffness of the commissure 330 compared to other portions of the leaflets. Other techniques and/or mechanisms can be used to form the commissure 330 and secure the commissure to a pair of window struts 138a, 138b of a frame, including any of those described in U.S. Pat. No. 9,393,110 (such as the commissures disclosed in any of FIGS. 63-71 of the '110 patent) and WIPO Publication No. WO2022/056048, which are incorporated herein by reference.

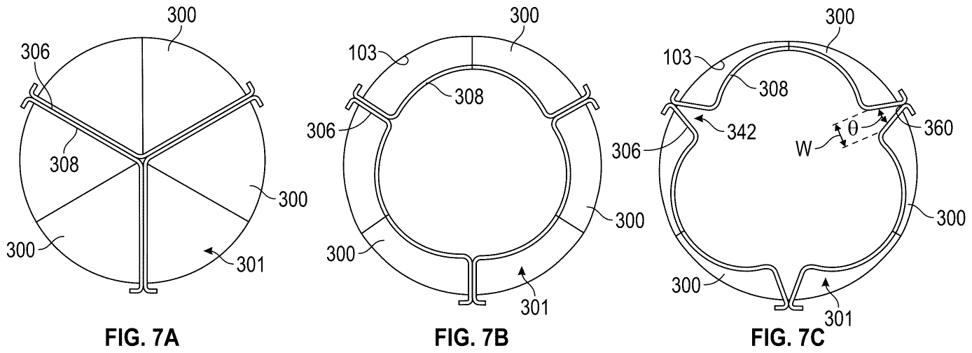

After inserting the commissure 330 into a commissure window 142 of the frame, a pivot axis 340 can be formed at the cut-out region 310 of each leaflet, extending through the nadir 316 of the corresponding notch 312. Although the pivot axis 340 depicted as being at an oblique angled relative to the coaptation edge region 308 of the flattened leaflet (see FIG. 5), in some examples, the commissure tab 306 can be pulled slightly upward when fully assembled and secured to a commissure window, which can cause the angle of the pivot axis 340 to change slightly. For example, when the leaflet assembly is in the closed state (see FIG. 7A below), the outflow edge 314 of the commissure tab 306 can be parallel or substantially parallel to the coaptation edge region 308 (and at about the same height) and perpendicular to a longitudinal axis of the prosthetic valve, as depicted in FIG. 6B. In the closed state, the pivot axis 340 can extend axially in parallel to the longitudinal axis of the prosthetic valve. When the leaflet assembly moves from the closed state to a partially open state (see FIG. 7B below), the coaptation edge region 308 can move slightly downward toward the inflow end of the frame (as indicated by the dashed line 308) and cause the pivot axis 340 to rotate accordingly. For example, the pivot axis 340 depicted in FIG. 6B is rotated in a counter-clockwise direction compared to the pivot axis depicted in FIG. 5 (and the pivot axis at the opposing tab will rotate in a clockwise direction). In some examples, when the leaflet assembly moves from the closed state to the partially open state, the pivot axis 340 can extend from the nadir 316 of the notch 312 to a nadir 325 of the recessed region 326. When the leaflet assembly moves between the partially open state and closed state, the coaptation edge region 308 of the leaflet 300 can pivot about the pivot axis 340 partially due to reduced leaflet material at the cut-out region 310 and partially due to increased stiffness at the commissure 330.

After inserting the commissure 330 into a commissure window 142 of the frame, a pivot axis 340 can be formed at the cut-out region 310 of each leaflet, extending through the nadir 316 of the corresponding notch 312. Although the pivot axis 340 depicted as being at an oblique angled relative to the coaptation edge region 308 of the flattened leaflet (see FIG. 5), in some examples, the commissure tab 306 can be pulled slightly upward when fully assembled and secured to a commissure window, which can cause the angle of the pivot axis 340 to change slightly. For example, when the leaflet assembly is in the closed state (see FIG. 7A below), the outflow edge 314 of the commissure tab 306 can be parallel or substantially parallel to the coaptation edge region 308 (and at about the same height) and perpendicular to a longitudinal axis of the prosthetic valve, as depicted in FIG. 6B. In the closed state, the pivot axis 340 can extend axially in parallel to the longitudinal axis of the prosthetic valve. When the leaflet assembly moves from the closed state to a partially open state (see FIG. 7B below), the coaptation edge region 308 can move slightly downward toward the inflow end of the frame (as indicated by the dashed line 308) and cause the pivot axis 340 to rotate accordingly. For example, the pivot axis 340 depicted in FIG. 6B is rotated in a counter-clockwise direction compared to the pivot axis depicted in FIG. 5 (and the pivot axis at the opposing tab will rotate in a clockwise direction). In some examples, when the leaflet assembly moves from the closed state to the partially open state, the pivot axis 340 can extend from the nadir 316 of the notch 312 to a nadir 325 of the recessed region 326. When the leaflet assembly moves between the partially open state and closed state, the coaptation edge region 308 of the leaflet 300 can pivot about the pivot axis 340 partially due to reduced leaflet material at the cut-out region 310 and partially due to increased stiffness at the commissure 330.

Note that in either the partially open state (FIG. 7B) or the fully open state (FIG. 7C), the coaptation edge regions 308 of the leaflets are spaced radially apart from the inner surface 103 of the frame 102. This is because after attaching the leaflet assembly 301 to the frame 102, the cut-out regions 310 of the leaflets extend into an interior space of the frame 102 so that the coaptation edge regions 308 are spaced radially apart from the inner surface 103 of the frame. For example, the notches 312 can space the coaptation edge regions 308 of the leaflets radially inwardly away from the inner surface 103 of the frame. Thus, as the leaflet assembly 301 moves from the closed state (FIG. 7A) to the partially open state (FIG. 7B), the leaflets 300 can pivot about the corresponding pivot axes 340, which offset or separate the coaptation edge regions 308 of the leaflets from the inner surface 103 of the frame. As a result, the likelihood of frame abrasion to the leaflets 300 can be reduced, and the durability of the leaflets 300 can be improved.

As the leaflet assembly 301 further opens, for example, from the partially open state (FIG. 7B) to the fully open state (FIG. 7C), portions of the commissure tabs 306 at each commissure can be configured to pivot relative to each other at respective pivot axes 338 (FIG. 6B) so as to form the V-shaped gap 342 therebetween. The pivot axes 338 of the commissure tabs 306 demarcates portions of the commissure tabs that are moveable and extend into the interior space of the frame and portions of the commissure tabs that are non-moveable and extend into the commissure window.

Thus, in some examples, the leaflets 300 can open according to the following sequence. First, the coaptation edge regions 308 pivot relative to the commissure tabs 306 at respective pivot axes 340 intersecting the cut-out regions 310 so that the coaptation edge regions 308 move apart from each other (for example, when moving from the closed state of FIG. 7A to the partially open state of FIG. 7B). Afterwards, the commissure tabs 306 at each commissure can pivot at the pivot axes 338 and further move apart from each other (for example, when moving from the partially open state of FIG. 7B to the fully open state of FIG. 7C). The pivot axes 340 can be referred to as primary pivot axes of the leaflets and the pivot axes 338 can be referred to as secondary pivot axes of the leaflets.

In some known prosthetic heart valves, offset pivot axes of the leaflets are accomplished by providing the commissure tabs with additional material that allows the commissure tabs to be folded form multiple layers of leaflet material that effectively offset the pivot axes of the coaptation edge regions from the inner surface of the frame. While effective to reduce leaflet abrasion, the contact between multiple layers of material at the commissures may promote tissue overgrowth or thrombus over the surfaces of the commissures, which in turn may result in relatively rigid regions of the leaflets that extend radially inward over time, and therefore may limit the effective orifice area of the valve in the open position of the leaflets. Additionally, folding of the commissure tabs in the formation of the commissures may increase the time and complexity required for assembling the valve. Advantageously, the leaflets 300 formed with notches or cut-out regions 310 can effectively offset the pivot axes of the coaptation edge regions away from the inner surface of the frame to reduce leaflet abrasion without additional folded leaflet layers forming the commissures, which can reduce the risk of tissue overgrowth or thrombus formation at the commissures. Moreover, valve assembly is simplified because less steps are required to form the commissures.

Additionally, the gap 342 formed between the commissure tabs 306 when the leaflet assembly is in the fully open state mimic the gaps formed when native leaflets open. The gap 342 can be sufficiently large to allow a sufficient amount of blood flow or “washout” between the commissure tabs, thereby further reducing the likelihood of tissue ingrowth and thrombus or pannus formation at the commissure regions. In certain examples, the largest width (W) spanned by the gap 342 ranges between 50 μm and 350 μm, or between 100 μm and 300 μm (for example, about 100 μm), all inclusive. In certain examples, the angle (θ) formed by the gap 342 can range between 5°and 20°, or between 10°and 12°(for example, about 11°), all inclusive.

In some cases, over a period of time, a small degree of tissue formation may occur at the root 360 of the commissure between the commissure tab 306, thereby forming a small tissue bridge between the commissure tabs. The tissue bridge can function as a natural leaflet separator that can maintain a degree of separation between the commissure tabs that promotes washout between the commissure tabs. The tissue bridge can be about 100 μm and 300 μm (for example, about 100 μm) in width and can extend up to about 0.5 mm away from the commissure root 360.

In the example depicted in FIG. 5, the leaflet 300 is generally V-shaped. Specifically, the cusp edge 304 has a generally V-shaped contour and the width of the leaflet 300 along the cusp edge 304 (measured horizontally as shown in FIG. 5) continuously decreases from the recessed region 326 toward an apex region 305 of the leaflet 300. FIG. 3 illustrates relative positions of the leaflet 300 and the frame 102 after mounting the leaflet 300 to the frame 102 (the outer perimeter of the leaflet 300 is shown in dashed lines), according to one example. As shown, the outflow edge 302 of the leaflet 300 can align with the outflow end portions 148 of the axial struts 140 and extend across multiple first row 120 of cells 118. In some examples, the outflow edge 302 can align with upper ends of the commissure windows 142. As described above, each commissure tab 306 of the leaflet 300 can be paired with an adjacent commissure tab of an adjacent leaflet to form a commissure of a leaflet assembly that is inserted into a corresponding commissure window 142 of the frame. Each side of the cusp edge 304 can extend along a substantially straight line and be attached to three interconnected angled struts 134, 132, and 130 located on three adjacent rows of the frame. The apex region 305 of leaflet 300 can align with the apex region 152 at the inflow end 108 of the frame 102.

Additional features of the leaflet 300 and some variants of the leaflet are described in the co-pending U.S. Provisional Application No., 64/448,767, filed Feb. 28, 2023, which is incorporated herein by reference.

### Exemplary Prosthetic Valves Having U-Shaped Leaflets

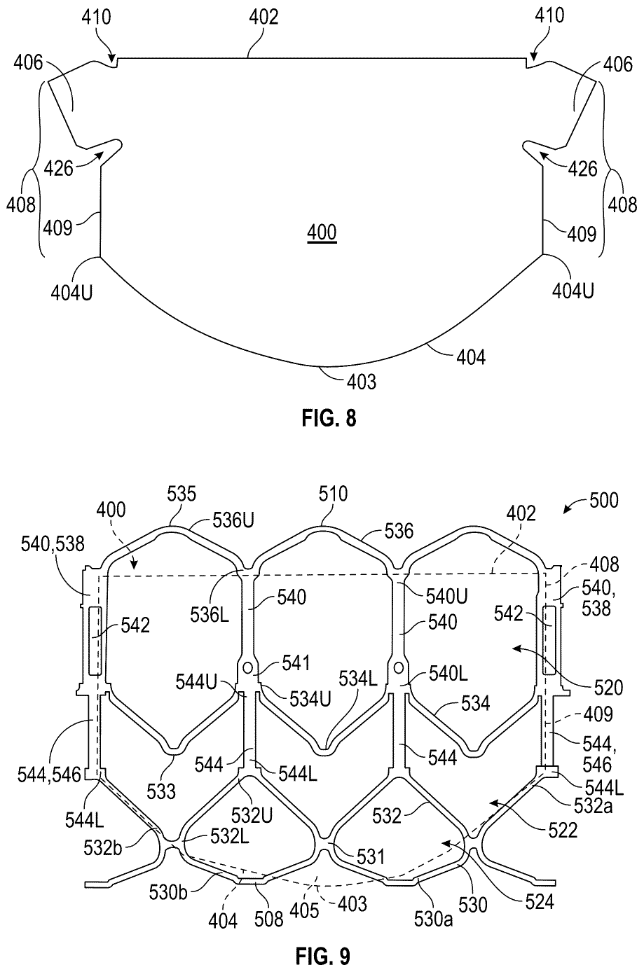

FIG. 8 shows a flattened view of a leaflet 400, according to another example. As shown, the leaflet 400 has an outflow edge 402 and a cusp edge 404 opposite the outflow edge 402. The leaflet 400 has two opposing side portions 408 extending between the outflow edge 402 and the cusp edge 404. Each side portion 408 includes a commissure tab 406 that is similar to the commissure tabs 306 of FIG. 5. Each commissure tab 406 can be connected to the outflow edge 402 by a cutout region 410 that is similar to the cut-out region 310 of FIG. 5.

The leaflet 400 is generally U-shaped. As shown in FIG. 8, each side portion 408 includes a sub-commissure side edge 409, which extends from an upper end 404U of the cusp edge 404 to a recessed region 426 which connects the sub-commissure side edge 409 to a corresponding commissure tab 406. While the cusp edge 404 has a rounded contour, each sub-commissure side edge 409 is substantially straight and perpendicular to the outflow edge 402. As such, the width of the leaflet 400 (measured horizontally as shown in FIG. 8) can remain substantially constant along the vertical length of the sub-commissure side edges 409 until reaching the upper end 404U of the cusp edge 404, where the width of the leaflet 400 starts to taper or progressively decrease toward an apex 403 of the leaflet 400.

A plurality of leaflets 400 can be assembled together to form a leaflet assembly (similar to the leaflet assembly 301) that is coupled to a frame of a prosthetic valve.

FIG. 9 shows a portion of a frame 500 to which the plurality of leaflets 400 can be attached. FIG. 9 also illustrates relative positions of the leaflet 400 and the frame 500 after mounting the leaflet 400 to the frame 500 (the outer perimeter of the leaflet 400 is shown in dashed lines). Similar to the frame 102, the frame 500 is radially compressible and expandable between a radially compressed configuration and a radially expanded configuration, and the frame 500 can be made of any of various suitable plastically-expandable materials or self-expanding materials.

As shown in FIG. 9, the frame 500 includes a plurality of circumferentially extending rows of angled struts. Specifically, the frame 500 includes a first row of angled struts 536 defining an outflow end 510 of the frame, a second row of angled struts 534 upstream of the first row of angled struts 536, a third row of angled struts 532 upstream of the second row of angled struts 534, and a fourth row of angled struts 530 upstream of the third row of angled struts 532. In this example, the fourth row of angled struts 530 defines an inflow end 508 of the frame.

The frame 500 includes a plurality of first axial frame members 540 (similar to the axial struts 140 of FIGS. 2-3) bridging the first row of angled struts 536 and the second row of angled struts 534. Additionally, the frame 500 includes a plurality of second axial frame members 544 bridging the second row of angled struts 534 and the third row of angled struts 532. The first axial frame members 540 and the second axial frame members 544 are substantially parallel to a central longitudinal axis of the frame 500. Each second axial frame member 544 can be axially aligned with a corresponding first axial frame member 540.

As shown in FIG. 9, each strut of the first row of angled struts 536 has an upper end 536U and a lower end 536L. The upper end 536U of each strut of the first row of angled struts 536 can be connected to the upper end 536U of an adjacent strut of the first row of angled struts 536 to form an outflow apex 535 of the frame 500. The lower end 536L of each strut of the first row of angled struts 536 can be connected to the lower end 536L of an adjacent strut of the first row of angled struts 536 and an upper end 540U of one of the first axial frame members 540.

Additionally, each strut of the second row of angled struts 534 has an upper end 534U and a lower end 534L. The upper end 534U of each strut of the second row of angled struts 534 can be connected to a junction 541 defined by a lower end 540L of one of the first axial frame members 540 and an upper end 544U of one of the second axial frame members 544. The lower end 534L of each strut of the second row of angled struts 534 can be connected to the lower end 534L of an adjacent strut of the second row of angled struts 534 to form a vertex 533 that points toward the inflow end 508 of the frame.

Further, each strut of the third row of angled struts 532 has an upper end 532U and a lower end 532L. The upper end 532U of each strut of the third row of angled struts 532 can be connected to a lower end 544L of one of the second axial frame members 544, and the lower end 532L of each strut of the third row of angled struts 532 can be connected to the lower end 532L of an adjacent strut of the third row of angled struts 532.

In some examples, there are exactly nine first axial frame members 540 and nine second axial frame members 544. In other examples, the number of first axial frame members 540 and the number of second axial frame members 544 can be more than nine or less than nine.

In some examples, as depicted in FIG. 9, there are exactly two angled struts in the second row of angled struts 534 and exactly two angled struts in the third row of angled struts 532 extending between two circumferentially adjacent second axial frame members 544. In other examples, there can be three or more angled struts in the second row of angled struts 534 or in the third row of angled struts 532 between two circumferentially adjacent second axial frame members 544.

As shown in FIG. 9, when the frame 500 is in the expanded configuration, each angled strut in the third row of angled struts 532 can be parallel to and axially aligned with a corresponding angled strut in the second row of angled struts 534.

Selected ones of the first axial frame members 540 include first leaflet attachment frame members 538, each of which can have a commissure window 542 (similar to the commissure window 142 defined by the window struts 138 of FIGS. 2-3). Selected ones of the second axial frame members 544 include second leaflet attachment frame members 546. Each second leaflet attachment frame member 546 is axially aligned with a corresponding first leaflet attachment frame member 538.

One or more (for example, two, as shown in FIG. 9) first axial frame members 540 without commissure windows 542 can be positioned between, in the circumferential direction, two first leaflet attachment frame members 538 comprising commissure windows 542.

When the plurality of leaflets 400 are mounted to the frame 500, each side portion 408 of each leaflet 400 can be attached to one of the first leaflet attachment frame members 538 (along a majority of a length thereof) and one of the second leaflet attachment frame members 546 (along a majority of a length thereof). Specifically, each commissure tab 406 of a leaflet 400 can be paired with an adjacent commissure tab of an adjacent leaflet to form a commissure that is attached to a corresponding first leaflet attachment frame member 538. The commissure tabs of each commissure can extend through the commissure window 542 of a corresponding first leaflet attachment frame member 538, similar to the example depicted in FIG. 6A. Additionally, each sub-commissure side edge 409 of each leaflet 400 can be attached to a corresponding second leaflet attachment frame member 546 (for example, via sutures). Each sub-commissure side edge 409 optionally can also be sutured to an adjacent sub-commissure side edge 409 of an adjacent leaflet 400.

In some examples, the cusp edge 404 of each leaflet 400 can extend from a first location at an upstream end (or lower end) of one of the second leaflet attachment frame members 546 to a second location at an upstream end (for example, or lower end) of another one of the second leaflet attachment frame members 546. For example, the two upper ends 404U of the cusp edge 404 can be respectively connected to the lower ends 544L of two adjacent second leaflet attachment frame members 546.

As shown in FIG. 9, the cusp edge 404 of each leaflet 400 can be attached (for example, via sutures) to two angled struts (for example, 532a, 532b) of the third row of angled struts 532, and each of the two angled struts (for example, 532a, 532b) can be connected to one of the second leaflet attachment frame members 546.

In some examples, the cusp edge 404 of each leaflet 400 can also be attached (for example, via sutures) to two angled struts (for example, 530a, 530b) of the fourth row of angled struts 530. For instance, the cusp edge 404 of each leaflet 400 can be directly sutured to the angled struts 530a, 530b. Alternatively, the cusp edge 404 of each leaflet 400 can be sutured to an inner skirt or a fabric reinforcing member, which in turn is sutured to the angled struts 530a, 530b. In other examples, the cusp edge 404 can be detached from any of the angled struts of the fourth row of angled struts 530.

In some examples, when the plurality of leaflets 400 are mounted to the frame 500, the apex 403 of the cusp edge 404 of each leaflet 400 can be located at the inflow end 508 of the frame 500. In some examples, as depicted in FIG. 9, an apex region 405 of each leaflet 400 is not connected to any strut of the frame 500. In such circumstances, the apex 403 of the leaflet 400 (for example, a midpoint of the cusp edge 404) can be located upstream of a junction 531 where two angled struts in the fourth row of angled struts 530 are connected to each other.

Similar to the frame 102, the frame 500 includes a plurality of open cells. For example, the first row of angled struts 536, the second row of angled struts 534, and the first axial frame members 540 can define a first row of first cells 520 of the frame; the second row of angled struts 534, the third row of angled struts 532, and the second axial frame members 544 can define a second row of second cells 522 of the frame; the third row of angled struts 532 and the fourth row of angled struts 530 can define a third row of third cells 524 of the frame. In some examples, the frame 500 includes nine first cells 520, nine second cells 522, and nine third cells 524.

As described herein, an upstream cell comprises at least one axial strut that is closer to the inflow end 508 of the frame than all axial struts of a downstream cell. Thus, the second row of second cells 522 are upstream of the first row of first cells 520, and the third row of third cells 524 are upstream of the second row of second cells 522.

As shown in FIG. 9, each first cell 520 and each second cell 522 can have six sides, and each third cell 524 can have four sides. Each first cell 520 has a convex hexagonal shape (that is, none of the interior angles of a first cell 520 is more than 180°) and each second cell 522 has a concave hexagonal shape (that is, at least one interior angle of a second cell 522 is greater than 180°). Each first cell 520 is stacked on top of (and axially aligned with) a corresponding second cell 522. Each third cell 524 is circumferentially located between two immediately adjacent second cells 522.

The size of each cell can be measured in area (enclosed by the struts), width (measured in the circumferential direction), and height or length (measured in the axial direction). In some examples, when the frame 500 is radially expanded, the area of each first cell 520 can be larger than the area of each second cell 522, and the area of each third cell 524 can be smaller than the area of each second cell 522. In some examples, the first cells 520 are longer than the second cells 522 in the axial direction of the frame. In some examples, the first cells 520, second cells 522, and third cells 524 have the same circumferential width.

As shown in FIG. 9, when the plurality of leaflets 400 are mounted to the frame 500, the outflow edge 402 of each leaflet 400 can align with the upper ends 540U of the first axial frame members 540 and extend across multiple first cells 520. In some examples, the outflow edge 402 can be upstream of the upper ends 540U and align with upper ends of the commissure windows 542. The two side portions 408 of each leaflet 400 can be respectively attached to two first leaflet attachment frame members 538 and two second leaflet attachment frame members 546, as described above. The cusp edge 404 of each leaflet 400 can be attached to two angled struts (for example, 532a, 532b) of the third row of angled struts 532. Optionally, the cusp edge 404 of each leaflet 400 can also be attached to two angled struts (for example, 530a, 530b) of the fourth row of angled struts 530. The apex region 405 of each leaflet 400 can extend freely between two adjacent third cells 524. As a result, the leaflets 400 can cover an entirety of the second row of second cells 522 and a majority area of the first row of first cells 520. In some examples, each leaflet 400 can also cover two third cells 524.

Compared to the leaflet 300 depicted in FIG. 5 (which is closer to a V-shape), the U-shaped leaflets 400 can have more leaflet material, especially in lower portions of the leaflets that are upstream of the commissures. For example, as shown in FIG. 3, after mounting the leaflets 300 to the frame 102, the cusp edges 304 of the leaflets 300 connects the commissures and extend in angular directions along respective angled struts in the second row of angled struts 134. In contrast, as shown in FIG. 9, after mounting the U-shaped leaflets 400 to the frame 500, immediately below the commissures are vertical sub-commissure side edges 409 which extend axially along the second leaflet attachment frame member 546. The cusp edges 404 of the leaflets 400 connect the lower ends 544L of the second leaflet attachment frame members 546 and extend in angular directions along respective angled struts in the third row of angled struts 532. Thus, the additional sub-commissure side edges 409 allow the leaflets 400 to have more leaflet material than the leaflets 300. Such additional leaflet material can create slack and contribute to the leaflets'movement between the open and closed states (FIGS. 7A-7C), thereby improving coaptation along the outflow edges 402 of the leaflets. Further, such additional leaflet material can improve the mobility of the leaflets 400 to facilitate blood washout from any pockets formed between the leaflets 400 and the outer skirt (for example, 106).

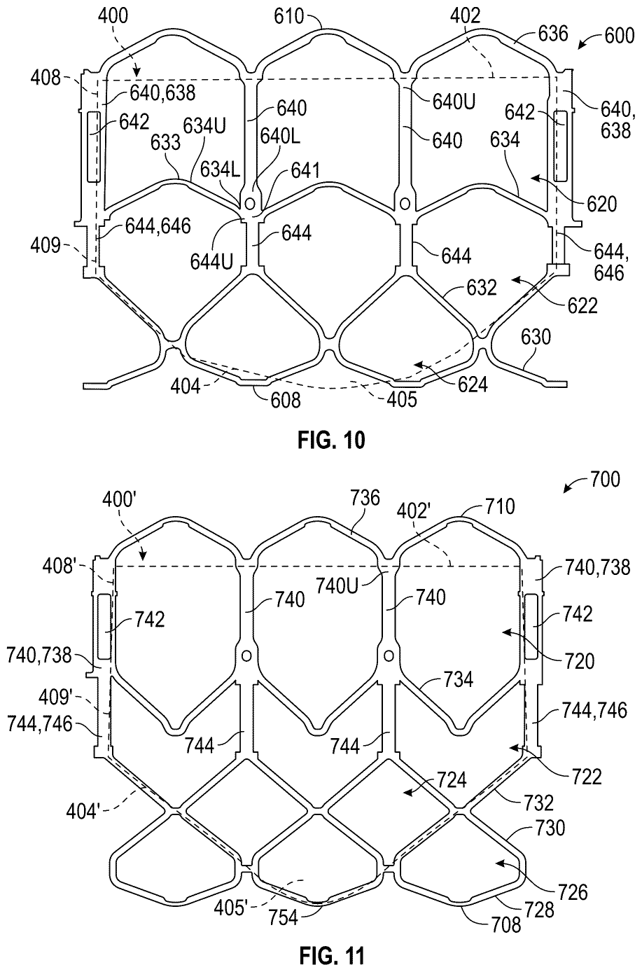

FIG. 10 shows a portion of another frame 600 to which the plurality of leaflets 400 can be attached. Similar to the frame 500, the frame 600 includes four rows of angled struts 636, 634, 632, and 630 circumferentially extending between an outflow end 610 and an inflow end 608 of the frame 600. Likewise, the frame 600 includes a plurality of first axial frame members 640 (similar to the first axial frame members 540) bridging the first row of angled struts 636 and the second row of angled struts 634, and a plurality of second axial frame members 644 bridging the second row of angled struts 634 and the third row of angled struts 632.

Similar to the frame 500, selected ones of the first axial frame members 640 include first leaflet attachment frame members 638, each of which can have a commissure window 642. Additionally, selected ones of the second axial frame members 644 include second leaflet attachment frame members 646 that are axially aligned with the corresponding first leaflet attachment frame members 638.

The second row of angled struts 634 of the frame 600 are oriented differently than the second row of angled struts 534 of the frame 500. Specifically, a lower end 634L of each strut of the second row of angled struts 634 can be connected to a junction 641 defined by a lower end 640L of one of the first axial frame members 640 and an upper end 644U of one of the second axial frame members 644. An upper end 634U of each strut of the second row of angled struts 634 can be connected to the upper end 634U of an adjacent strut of the second row of angled struts 634 to form a vertex 633 that points toward the outflow end 610 of the frame.

As shown in FIG. 10, the first row of angled struts 636, the second row of angled struts 634, and the first axial frame members 640 can define a first row of first cells 620; the second row of angled struts 634, the third row of angled struts 632, and the second axial frame members 644 can define a second row of second cells 622; the third row of angled struts 632 and the fourth row of angled struts 630 can define a third row of third cells 624 of the frame. In this example, the first and second cells 620, 622 are six-sided and the third cells 624 are four-sided. In contrast to the frame 500, each first cell 620 has a concave hexagonal shape and each second cell 622 has a convex hexagonal shape. In some examples, the area of each first cell 620 is larger than the area of each second cell 622, which is larger than the area of each third cell 624.

As shown in FIG. 10, when the plurality of leaflets 400 are mounted to the frame 600, the outflow edge 402 of each leaflet 400 can align with upper ends 640U of the first axial frame members 640 and extend across multiple first cells 620. In some examples, the outflow edge 402 can be upstream of the upper ends 640U and align with upper ends of the commissure windows 642. The two side portions 408 of each leaflet 400 can be respectively attached to two first leaflet attachment frame members 638 and two second leaflet attachment frame members 646. The cusp edge 404 of each leaflet 400 can be attached to two angled struts of the third row of angled struts 632. Optionally, the cusp edge 404 of each leaflet 400 can also be attached to two angled struts of the fourth row of angled struts 630. The apex region 405 of each leaflet 400 may not be connected to any strut of the frame 600, and thus can extend freely between two adjacent third cells 624.

FIG. 11 shows a portion of another frame 700 to which a plurality of leaflets 400′ can be attached. Each leaflet 400′ is similar to the leaflet 400 except that the leaflet 400′ can be larger and have longer cusp edges 404′ than the leaflet 400. The leaflet 400′ can be attached to frames that are larger than the frame 500, for example, frames having five or more rows of angled struts.

For example, FIG. 11 shows that the frame 700 includes five rows of angled struts 736, 734, 732, 730, and 728 circumferentially extending between an outflow end 710 and an inflow end 708 of the frame 700. The frame 700 also includes a plurality of first axial frame members 740 (similar to the first axial frame members 540) bridging the first row of angled struts 736 and the second row of angled struts 734, and a plurality of second axial frame members 744 bridging the second row of angled struts 734 and the third row of angled struts 732.

Similar to the frame 500, selected ones of the first axial frame members 740 include first leaflet attachment frame members 738, each of which can have a commissure window 742. Additionally, selected ones of the second axial frame members 744 include second leaflet attachment frame members 746 that are axially aligned with the corresponding first leaflet attachment frame members 738.

As shown in FIG. 11, the first row of angled struts 736, the second row of angled struts 734, and the first axial frame members 740 can define a first row of first cells 720; the second row of angled struts 734, the third row of angled struts 732, and the second axial frame members 744 can define a second row of second cells 722; the third row of angled struts 732 and the fourth row of angled struts 730 can define a third row of third cells 724; and the fourth row of angled struts 730 and the fifth row of angled struts 728 can define a fourth row of fourth cells 726. In this example, the first and second cells 720, 722 are six-sided and the third and fourth cells 724, 726 are four-sided.

As shown in FIG. 11, when the plurality of leaflets 400′ are mounted to the frame 700, the outflow edge 402′ of each leaflet 400′ can align with upper ends 740U of the first axial frame members 740 and extend across multiple first cells 720. In some examples, the outflow edge 402′ can be upstream of the upper ends 740U and align with upper ends of the commissure windows 742. The two side portions 408′ of each leaflet 400′ can be respectively attached to two first leaflet attachment frame members 738 and two second leaflet attachment frame members 746. Different from the example depicted in FIG. 9, the longer cusp edge 404′ of each leaflet 400′ can be attached to two angled struts of the third row of angled struts 732, two angled struts of the fourth row of angled struts 730, and two angled struts of the fifth row of angled struts 728. In some examples, the apex region 405′ of each leaflet 400′ can be connected to an apex 754 formed by two angled struts of the fifth row of angled struts 728. In this example, the leaflets 400′ can cover an entirety of the second row of second cells 722 and a majority area of the first row of first cells 720. Additionally, each leaflet 400′ can completely cover two third cells 724 and one fourth cell 726.

### Exemplary Delivery Techniques

For implanting a prosthetic valve within the native aortic valve via a transfemoral delivery approach, the prosthetic valve can be mounted in a radially compressed state along the distal end portion of a delivery apparatus. The prosthetic valve and the distal end portion of the delivery apparatus can be inserted into a femoral artery and then advanced into and through the descending aorta, around the aortic arch, and through the ascending aorta. The prosthetic valve can be positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) can be introduced into the left ventricle through a surgical opening in the chest and the apex of the heart and the prosthetic valve can be positioned within the native aortic valve. Alternatively, in a transaortic procedure, a prosthetic valve (on the distal end portion of the delivery apparatus) can be introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, into the right atrium, across the atrial septum (for example, through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) can be introduced into the left ventricle through a surgical opening in the chest and the apex of the heart and the prosthetic valve can be positioned within the native mitral valve.

For implanting a prosthetic valve within the native tricuspid valve, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, and into the right atrium, and the prosthetic valve can be positioned within the native tricuspid valve. A similar approach can be used for implanting the prosthetic valve within the native pulmonary valve or the pulmonary artery, except that the prosthetic valve can be advanced through the native tricuspid valve into the right ventricle and toward the pulmonary valve/pulmonary artery.

Another delivery approach is a trans-atrial approach whereby a prosthetic valve (on the distal end portion of the delivery apparatus) can be inserted through an incision in the chest and an incision made through an atrial wall (of the right or left atrium) for accessing any of the native heart valves. Atrial delivery can also be made intravascularly, such as from a pulmonary vein. Still another delivery approach is a trans-ventricular approach whereby a prosthetic valve (on the distal end portion of the delivery apparatus) can be inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Exemplary Expansion Mechanisms of Prosthetic Valves

For any of the prosthetic valves described herein, the frame can be made of any of various suitable plastically-expandable materials. When constructed of a plastically-expandable material, the frame (and thus the prosthetic valve) can be crimped to a radially collapsed configuration on a delivery catheter or apparatus and then expanded inside a patient by an inflatable balloon or equivalent expansion mechanism. In certain examples, during delivery to the implantation site, the prosthetic valve can be placed inside of a delivery capsule or sheath to protect against the prosthetic valve contacting the patient's vasculature, such as when the prosthetic valve is advanced through a femoral artery. The capsule can also retain the prosthetic valve in a radially compressed state having a slightly smaller diameter and crimp profile than may be otherwise possible without a capsule by preventing any recoil (expansion) of the frame once it is crimped onto the delivery apparatus.

Suitable plastically-expandable materials that can be used to form the frame include, without limitation, stainless steel, a biocompatible, high-strength alloys (for example, a cobalt-chromium or a nickel-cobalt-chromium alloys), polymers, or combinations thereof. In particular examples, frame is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight.

Additional details on balloon expandable prosthetic valves can be found in U.S. Pat. No. 9,393,110, and U.S. Provisional Application Nos. 63/178,416, filed Apr. 22, 2021, 63/194,830, filed May 28, 2021, and 63/279,096, filed Nov. 13, 2021, all of which are incorporated by reference herein.

Any of the prosthetic valves described herein can be self-expandable. For example, the frame of the prosthetic valve can comprise a shape-memory material (for example, Nitinol). When the prosthetic valve is self-expandable, the frame (and thus the prosthetic valve) can be crimped to a radially compressed configuration and restrained in the compressed configuration by insertion into a sheath or equivalent mechanism of a delivery catheter. Once inside the body at the desired implantation site, the prosthetic valve can be deployed or released from the delivery sheath, which allows the prosthetic valve to expand to its functional size. In some examples, the frame (and therefore the prosthetic valve) can partially self-expand from the radially compressed configuration to a partially radially expanded configuration. The frame (and therefore the prosthetic valve) can be further radially expanded from the partially expanded configuration to a further radially expanded configuration via one or more actuation assemblies (for example, an inflatable balloon and/or one or more mechanical actuators) of the delivery apparatus.

Additional details regarding exemplary self-expandable prosthetic valves and the related delivery apparatus/catheters/systems are described in U.S. Pat. Nos. 8,652,202, 9,155,619, and 9,867,700, all of which are incorporated herein by reference.

Additionally, and/or alternatively, any of the prosthetic valves described herein can be mechanically expandable. For example, the struts of the frame can be pivotably coupled to one another at one or more pivot joints along the length of each strut. As a result, an axial force applied to the frame (for example, pressing the inflow end and the outflow end of the frame toward each other or pulling the inflow end and the outflow end of the frame away from each other) can cause the prosthetic valve to radially expand or compress. The axial force can be generated by actuating one or more mechanical actuators of the delivery apparatus that are operatively coupled to the frame.

Additional details regarding exemplary mechanically-expandable prosthetic valves and the related delivery apparatus/catheters/systems are described in U.S. Patent Application Publication Nos. 2018/0153689, 2018/0311039, 2019/0060057, and PCT Patent Application Publication No. WO/2021/188476, all of which are incorporated by reference herein.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising: an annular frame that is radially collapsible to a collapsed configuration and radially expandable to an expanded configuration, the annular frame comprising an inflow end and an outflow end; and a leaflet structure positioned within the annular frame and configured to permit blood flow from the inflow end to the outflow end and block blood fluid flow from the outflow end to the inflow end, wherein the leaflet structure comprises a plurality of leaflets, wherein the annular frame comprises: a first row of angled struts defining the outflow end; a second row of angled struts upstream of the first row of angled struts; a third row of angled struts upstream of the second row of angled struts; a plurality of first axial frame members bridging the first row of angled struts and the second row of angled struts; and a plurality of second axial frame members bridging the second row of angled struts and the third row of angled struts, wherein the first row of angled struts, the second row of angled struts, and the first axial frame members define a first row of first cells, wherein the first cells are six-sided cells; wherein the second row of angled struts, the third row of angled struts, and the second axial frame members define a second row of second cells upstream of the first row of first cells, wherein the second cells are six-sided cells; wherein each leaflet comprises an outflow edge, a cusp edge opposite the outflow edge, and two opposing side portions extending between the outflow edge and the cusp edge, wherein selected ones of the first axial frame members comprise first leaflet attachment frame members and selected ones of the second axial frame members comprise second leaflet attachment frame members, wherein each side portion of each leaflet is attached to one of the first leaflet attachment frame members and one of the second leaflet attachment frame members.

Example 2. The prosthetic valve of any example herein, particularly example 1, wherein each side portion of each leaflet comprises a commissure tab and a sub-commissure side edge, wherein each commissure tab is paired with an adjacent commissure tab of an adjacent leaflet to form a commissure that is attached to a corresponding first leaflet attachment frame member.

Example 3. The prosthetic valve of any example herein, particularly example 2, wherein the commissure tabs of each commissure extend through a commissure window of a corresponding first leaflet attachment frame member.

Example 4. The prosthetic valve of any example herein, particularly any one of examples 2-3, wherein each sub-commissure side edge of each leaflet is attached to a corresponding second leaflet attachment frame member.

Example 5. The prosthetic valve of any example herein, particularly any one of examples 1-4, wherein there are exactly nine first axial frame members and nine second axial frame members.

Example 6. The prosthetic valve of any example herein, particularly any one of examples 1-5, wherein each second axial frame member is axially aligned with a corresponding first axial frame member.

Example 7. The prosthetic valve of any example herein, particularly any one of examples 1-6, wherein the cusp edge of each leaflet is attached to two angled struts of the third row of angled struts, wherein each of the two angled struts is connected to one of the second leaflet attachment frame members.

Example 8. The prosthetic valve of any example herein, particularly example 7, wherein the cusp edge of each leaflet comprises an apex region that is not connected to any strut of the frame.

Example 9. The prosthetic valve of any example herein, particularly any one of examples 1-8, wherein there are exactly two angled struts in the second row of angled struts extending between two circumferentially adjacent second axial frame members.

Example 10. The prosthetic valve of any example herein, particularly any one of examples 1-9, wherein there are exactly two angled struts in the third row of angled struts extending between two circumferentially adjacent second axial frame members.

Example 11. The prosthetic valve of any example herein, particularly any one of examples 1-10, wherein when the frame is in the expanded configuration, each angled strut in the third row of angled struts is parallel to and circumferentially aligned with a corresponding angled strut in the second row of angled struts.

Example 12. The prosthetic valve of any example herein, particularly any one of examples 1-11, wherein the first cells are larger than the second cells when the frame is in the expanded configuration.

Example 13. The prosthetic valve of any example herein, particularly example 12, wherein when the frame is in the expanded configuration, each first cell has a convex hexagonal shape and each second cell has a concave hexagonal shape.

Example 14. The prosthetic valve of any example herein, particularly any one of examples 12-13, wherein the frame further comprises a fourth row of angled struts upstream of the third row of angled struts, wherein the fourth row of angled struts and the third row of angled struts define a third row of third cells, wherein the third cells are a four-sided cells.

Example 15. The prosthetic valve of any example herein, particularly example 14, wherein the fourth row of angled struts define the inflow end of the frame.

Example 16. The prosthetic valve of any example herein, particularly any one of examples 14-15, wherein the third cells are smaller than the second cells.

Example 17. The prosthetic valve of any example herein, particularly any one of examples 14-16, wherein a midpoint of the cusp edge of each leaflet is located upstream of a junction where two angled struts in the fourth row of angled struts are connected to each other.

Example 18. The prosthetic valve of any example herein, particularly any one of examples 1-13, wherein the frame further comprises two or more rows of angled struts upstream of the third row of angled struts.

Example 19. The prosthetic valve of any example herein, particularly example 18, wherein the two or more rows of angled struts upstream of the third row of angled struts form one or more rows of four-sided cells.

Example 20. The prosthetic valve of any example herein, particularly any one of examples 1-19, wherein the first and second axial frame members are parallel to a central longitudinal axis of the frame.

Example 21. A prosthetic valve, comprising: a radially expandable and compressible frame comprising an inflow end and an outflow end; and a leaflet assembly comprising a plurality of leaflets secured to the frame, wherein the frame comprises: a first row of angled struts defining the outflow end of the frame; a second row of angled struts upstream of the first row of angled struts; a third row of angled struts upstream of the second row of angled struts; a fourth row of angled struts upstream of the third row of angled struts; a plurality of first axial frame members bridging the first row of angled struts and the second row of angled struts; and a plurality of second axial frame members bridging the second row of angled struts and the third row of angled struts; wherein selected ones of the first axial frame members comprise first leaflet attachment frame members and selected ones of the second axial frame members comprise second leaflet attachment frame members; wherein each leaflet comprises an outflow edge, a curved cusp edge opposite the outflow edge, and two opposing side portions extending between the outflow edge and the cusp edge, wherein each side portion of each leaflet is attached to one of the first leaflet attachment frame members along a majority of a length thereof and to one of the second leaflet attachment frame members along a majority of a length thereof, wherein the cusp edge of each leaflet extends from a first location at an upstream end of one of the second leaflet attachment frame members to a second location at an upstream end of another one of the second leaflet attachment frame members.

Example 22. The prosthetic valve of any example herein, particularly example 21, wherein each side portion of each leaflet comprises a commissure tab and a sub-commissure side edge, wherein each commissure tab is paired with an adjacent commissure tab of an adjacent leaflet to form a commissure that is attached to a corresponding first leaflet attachment frame member.

Example 23. The prosthetic valve of any example herein, particularly example 22, wherein the commissure tabs of each commissure extend through a commissure window of a corresponding first leaflet attachment member.

Example 24. The prosthetic valve of any example herein, particularly any one of examples 21-23, wherein each sub-commissure side edge of each leaflet is attached to a corresponding second leaflet attachment frame member.

Example 25. The prosthetic valve of any example herein, particularly any one of examples 21-24, wherein the cusp edge of each leaflet tapers in width from the side portions to an apex of the cusp edge.

Example 26. The prosthetic valve of any example herein, particularly example 25, wherein the cusp edge of each leaflet is attached to two angled struts of the third row of angled struts.

Example 27. The prosthetic valve of any example herein, particularly any one of examples 25-26, wherein the apex of the cusp edge of each leaflet is located at the inflow end of the frame.

Example 28. The prosthetic valve of any example herein, particularly any one of examples 22-27, wherein the first row of angled struts, the second row of angled struts, and the first axial frame members define a first row of first cells of the frame, wherein the second row of angled struts, the third row of angled struts, and the second axial frame members define a second row of second cells of the frame, wherein each first cell and each second cell has six sides.

Example 29. The prosthetic valve of any example herein, particularly example 28, wherein the first cells are larger than the second cells when the frame is radially expanded.

Example 30. The prosthetic valve of any example herein, particularly example 29, wherein the first cells are longer than the second cells in an axial direction of the frame when the frame is radially expanded.

Example 31. The prosthetic valve of any example herein, particularly any one of examples 28-30, wherein the third row of angled struts and the fourth row of angled struts define a third row of third cells of the frame, wherein each third cell has four sides.

Example 32. The prosthetic valve of any example herein, particularly example 31, wherein the third cells are smaller than the first and second cells.

Example 33. The prosthetic valve of any example herein, particularly any one of examples 21-32, wherein the fourth row of angled struts defines the inflow end of the frame.

Example 34. A prosthetic valve, comprising: a radially expandable and compressible frame comprising an inflow end and an outflow end; and a leaflet assembly comprising a plurality of leaflets secured to the frame, wherein the frame comprises: a first row of first cells defining the outflow end of the frame, wherein the first cells are six-sided cells; a second row of second cells upstream of the first row of first cells, wherein the second cells are six-sided cells and are smaller than the first cells; and a third row of third cells upstream of the second row of second cells, wherein the third cells are four-sided cells and are smaller than the second cells.

Example 35. The prosthetic valve of any example herein, particularly example 34, wherein the first, second, and third cells have the same width.

Example 36. The prosthetic valve of any example herein, particularly any one of examples 34-35, wherein the third row of third cells defines the inflow end of the frame.

Example 37. The prosthetic valve of any example herein, particularly any one of examples 34-36, wherein the leaflets cover an entirety of the second row of second cells.

Example 38. The prosthetic valve of any example herein, particularly any one of examples 34-37, wherein the frame comprises a first row of angled struts, a second row of angled struts upstream of the first row of angled struts, and a plurality of first axial frame members bridging the first and second rows of angled struts, wherein the first row of angled struts, the second row of angled struts, and the plurality of first axial frame members define the first row of first cells.

Example 39. The prosthetic valve of any example herein, particularly example 38, wherein the frame comprises a third row of angled struts upstream of the second row of angled struts and a plurality of second axial frame members bridging the second and third rows of angled struts, wherein the second row of angled struts, the third row of angled struts, and the plurality of second axial frame members define the second row of second cells.

Example 40. The prosthetic valve of any example herein, particularly example 39, wherein the frame comprises a fourth row of angled struts upstream of the third row of angled struts, wherein the third and fourth rows of angled struts define the third row of third cells.

Example 41. The prosthetic valve of any example herein, particularly any one of examples 38-40, wherein each strut of the first row of angled struts has an upper end and a lower end, wherein the upper end of each strut of the first row of angled struts is connected to the upper end of an adjacent strut of the first row of angled struts to form an outflow apex of the frame, and wherein the lower end of each strut of the first row of angled struts is connected to the lower end of an adjacent strut of the first row of angled struts and an upper end of one of the first axial frame members.

Example 42. The prosthetic valve of any example herein, particularly any one of examples 38-41, wherein each strut of the second row of angled struts has an upper end and a lower end, wherein the upper end of each strut of the second row of angled struts is connected to a junction defined by a lower end of one of the first axial frame members and an upper end of one of the second axial frame members, and wherein the lower end of each strut of the second row of angled struts is connected to the lower end of an adjacent strut of the second row of angled struts to form a vertex that points toward the inflow end of the frame.

Example 43. The prosthetic valve of any example herein, particularly any one of examples 39-42, wherein each strut of the third row of angled struts has an upper end and a lower end, wherein the upper end of each strut of the third row of angled struts is connected to a lower end of one of the second axial frame members, and the lower end of each strut of the third row of angled struts is connected to the lower end of an adjacent strut of the third row of angled struts.

Example 44. The prosthetic valve of any example herein, particularly any one of examples 39-43, wherein each leaflet comprises an outflow edge, a cusp edge opposite the outflow edge, and two opposing side portions extending between the outflow edge and the cusp edge.

Example 45. The prosthetic valve of any example herein, particularly example 44, wherein each side portion of each leaflet is connected to one of the first axial frame members and one of the second axial frame members.

Example 46. The prosthetic valve of any example herein, particularly example 45, wherein each side portion of each leaflet is connected to one of the first axial frame members and one of the second axial frame members with sutures.

Example 47. The prosthetic valve of any example herein, particularly any one of examples 45-46, wherein the cusp edge of each leaflet is sutured to at least two struts of the third row of angled struts.

Example 48. A method comprising: delivering a prosthetic device in a radially compressed state to a target location; and radially expanding the prosthetic device to a radially expanded state, wherein the prosthetic device is a prosthetic valve according to any example herein, particularly any one of examples 1-47.

Example 49. A method comprising sterilizing the prosthetic valve of any example herein, particularly any one of examples 1-47.

The features described herein with regard to any example can be combined with other features described in any one or more of the other examples, unless otherwise stated. For example, any one or more of the features of one prosthetic valve can be combined with any one or more features of another prosthetic valve.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A prosthetic valve, comprising:
an annular frame that is radially collapsible to a collapsed configuration and radially expandable to an expanded configuration, the annular frame comprising an inflow end and an outflow end; and
a leaflet structure positioned within the annular frame and configured to permit blood flow from the inflow end to the outflow end and block blood fluid flow from the outflow end to the inflow end, wherein the leaflet structure comprises a plurality of leaflets,
wherein the annular frame comprises:
a first row of angled struts defining the outflow end;
a second row of angled struts upstream of the first row of angled struts;
a third row of angled struts upstream of the second row of angled struts;
a plurality of first axial frame members bridging the first row of angled struts and the second row of angled struts; and
a plurality of second axial frame members bridging the second row of angled struts and the third row of angled struts,
wherein the first row of angled struts, the second row of angled struts, and the first axial frame members define a first row of first cells, wherein the first cells are six-sided cells;
wherein the second row of angled struts, the third row of angled struts, and the second axial frame members define a second row of second cells upstream of the first row of first cells, wherein the second cells are six-sided cells;
wherein each leaflet comprises an outflow edge, a cusp edge opposite the outflow edge, and two opposing side portions extending between the outflow edge and the cusp edge,
wherein selected ones of the first axial frame members comprise first leaflet attachment frame members and selected ones of the second axial frame members comprise second leaflet attachment frame members,
wherein each side portion of each leaflet is attached to one of the first leaflet attachment frame members and one of the second leaflet attachment frame members.

2. The prosthetic valve of claim 1, wherein each side portion of each leaflet comprises a commissure tab and a sub-commissure side edge, wherein each commissure tab is paired with an adjacent commissure tab of an adjacent leaflet to form a commissure that is attached to a corresponding first leaflet attachment frame member.

3. The prosthetic valve of claim 2, wherein the commissure tabs of each commissure extend through a commissure window of a corresponding first leaflet attachment frame member.

4. The prosthetic valve of claim 2, wherein each sub-commissure side edge of each leaflet is attached to a corresponding second leaflet attachment frame member.

5. The prosthetic valve of claim 1, wherein there are exactly nine first axial frame members and nine second axial frame members.

6. The prosthetic valve of claim 1, wherein each second axial frame member is axially aligned with a corresponding first axial frame member.

7. The prosthetic valve of claim 1, wherein the cusp edge of each leaflet is attached to two angled struts of the third row of angled struts, wherein each of the two angled struts is connected to one of the second leaflet attachment frame members.

8. The prosthetic valve of claim 7, wherein the cusp edge of each leaflet comprises an apex region that is not connected to any strut of the frame.

9. The prosthetic valve of claim 1, wherein the frame further comprises two or more rows of angled struts upstream of the third row of angled struts.

10. The prosthetic valve of claim 1, wherein the first and second axial frame members are parallel to a central longitudinal axis of the frame.

11. A prosthetic valve, comprising:
a radially expandable and compressible frame comprising an inflow end and an outflow end; and
a leaflet assembly comprising a plurality of leaflets secured to the frame,
wherein the frame comprises:
a first row of angled struts defining the outflow end of the frame;
a second row of angled struts upstream of the first row of angled struts;
a third row of angled struts upstream of the second row of angled struts;
a fourth row of angled struts upstream of the third row of angled struts;
a plurality of first axial frame members bridging the first row of angled struts and the second row of angled struts; and
a plurality of second axial frame members bridging the second row of angled struts and the third row of angled struts;
wherein selected ones of the first axial frame members comprise first leaflet attachment frame members and selected ones of the second axial frame members comprise second leaflet attachment frame members;
wherein each leaflet comprises an outflow edge, a curved cusp edge opposite the outflow edge, and two opposing side portions extending between the outflow edge and the cusp edge,
wherein each side portion of each leaflet is attached to one of the first leaflet attachment frame members along a majority of a length thereof and to one of the second leaflet attachment frame members along a majority of a length thereof,
wherein the cusp edge of each leaflet extends from a first location at an upstream end of one of the second leaflet attachment frame members to a second location at an upstream end of another one of the second leaflet attachment frame members.

12. The prosthetic valve of claim 11, wherein each side portion of each leaflet comprises a commissure tab and a sub-commissure side edge, wherein each commissure tab is paired with an adjacent commissure tab of an adjacent leaflet to form a commissure that is attached to a corresponding first leaflet attachment frame member.

13. The prosthetic valve of claim 12, wherein the commissure tabs of each commissure extend through a commissure window of a corresponding first leaflet attachment member.

14. The prosthetic valve of claim 11, wherein each sub-commissure side edge of each leaflet is attached to a corresponding second leaflet attachment frame member.

15. The prosthetic valve of claim 12, wherein the first row of angled struts, the second row of angled struts, and the first axial frame members define a first row of first cells of the frame, wherein the second row of angled struts, the third row of angled struts, and the second axial frame members define a second row of second cells of the frame, wherein each first cell and each second cell has six sides.

16. The prosthetic valve of claim 15, wherein the first cells are larger than the second cells when the frame is radially expanded.

17. The prosthetic valve of claim 16, wherein the first cells are longer than the second cells in an axial direction of the frame when the frame is radially expanded.

18. The prosthetic valve of claim 15, wherein the third row of angled struts and the fourth row of angled struts define a third row of third cells of the frame, wherein each third cell has four sides.

19. A prosthetic valve, comprising:
a radially expandable and compressible frame comprising an inflow end and an outflow end; and
a leaflet assembly comprising a plurality of leaflets secured to the frame,
wherein the frame comprises:
a first row of first cells defining the outflow end of the frame, wherein the first cells are six-sided cells;
a second row of second cells upstream of the first row of first cells, wherein the second cells are six-sided cells and are smaller than the first cells; and
a third row of third cells upstream of the second row of second cells, wherein the third cells are four-sided cells and are smaller than the second cells.

20. The prosthetic valve of claim 19, wherein the leaflets cover an entirety of the second row of second cells.
